(19) United States
(12) Patent Application Publication
(10) Pub. No.: US 2010/0215696 A1
(43) Pub. Date: Aug. 26, 2010

(54) EFFICIENTLY REPLICABLE HEPTITIS C VIRUS MUTANT, A HEPTITIS C VIRUS MUTANT COMPRISING REPORTER GENE, A METHOD OF PREPARING OF HCV VACCINE USING THE SAME AND A METHOD OF SCREENING ANTI HCV COMPOSITION USING THE SAME

(76) Inventors: Sung-Key Jang, Pohang-city (KR); Chon Saeng Kim, Iksan-shi (JP)

Correspondence Address: JHK LAW, P.O. BOX 1078, LA CANADA, CA, 91012-1078, US

(21) Appl. No.: 12/597,747

(22) PCT Filed: Apr. 28, 2008
(86) PCT No.: PCT/KR08/02405

§ 371 Date: Oct. 26, 2009

Related U.S. Application Data
(60) Provisional application No. 60/914,667, filed on Apr. 27, 2007.

(51) Int. Cl. A61K 39/29; C12N 15/51; C12N 7/01; C12N 15/63; C12N 5/00; C07K 16/00; C12Q 1/70; A61K 48/00; A61K 35/76; A61P 31/12

(52) U.S. Cl. 424/228.1; 536/23.72; 435/235.1; 435/320.1; 435/325; 530/389.4; 435/5; 514/44 R; 424/93.6

(57) ABSTRACT

The present invention relates an efficiently replicating a modified hepatitis virus (HCV) mutant, and a modified HCV further comprising reporter gene, a method of preparing HCV vaccine using the same, and a method of screening anti-HCV material using the same. The present invention is to overcome the defect that the conventional HCV cell culture systems are unable to produce a sufficient amount of virus, thereby causing it difficult to efficiently induce or measure HCV infection. Because the present invention can allow production of HCV in a large amount an efficiently observing HCV infection in a living cell, it can make it possible to achieve many studies that were previously highly challenging, including studies on infection routes, and assembly and release of HCV. In addition, the present invention contributes to studies for searching anti-HCV agents being inhibiting all stages of the HCV life cycle, not being limited to HCV replication.

### BACKGROUND OF THE INVENTION

(a) Field of the Invention

The present invention relates an efficiently replicating modified hepatitis C virus (hereinafter “HCV”), and a modified HCV further including a reporter gene, a method of preparing an HCV vaccine using the same, and a method of screening an anti-HCV material using the same.

(b) Description of the Related Art

It is estimated that 170 million individuals worldwide are chronically infected with HCV. Most acute HCV infections progress to be chronic, which may eventually lead to liver diseases such as chronic hepatitis, liver cirrhosis, and hepatocellular carcinoma. A protective vaccine does not yet exist, and therapeutic options are limited. Interferon-alpha (IFN-α) in combination with Ribavirin is the only therapy that is currently recommended as appropriate. However, it is reported that the therapy is still ineffective for more than half of infected patients, it requires long period of treatments, and it is accompanied by various side effects. This background shows that effective therapies and vaccines for HCV infections need to be developed.

The availability of a cell-culture system is a prerequisite to studying HCV and devising strategies for prophylactic and therapeutic treatments of HCV infections. Thus, it is necessary to secure a simple system in which the steps of formation, release, and infection of new cells are sufficiently imitated, in other words, a cell-culture-based HCV replication system.

One of the most recent achievements in cell-culture-based HCV systems is a virus production system that is based on the transfection of the human hepatoma cell line Huh 7 with genomic HCV RNA (JFH1) isolated from a patient with fulminant hepatitis. This model has allowed studying all stages of the HCV life cycle, and in fact many studies on HCV infection are being performed based on this model.

However, the usefulness of the above virus production system is limited in that only limited virus yields have been possible from the system. Since studies to find therapeutic interventions and to develop vaccines require a significant amount of virus, the above virus production system falls short for effectively performing quantitative assays and studying cells infected with the virus.

Furthermore, it is necessary to secure a system that can identify (detect) materials with anti-HCV effects or verify efficacies of potential anti-HCV agents. In particular, it is necessary to develop a system by which a mutant that facilitates virus replication can be identified or a system by which HCV infection can be quantified with a heterologous sequence inserted in the virus.

### SUMMARY OF THE INVENTION

The present invention is to overcome the defect that the conventional HCV cultivation systems are unable to produce a sufficient amount of virus, such that it is difficult to effectively cause HCV infection and to quantify the infection. Therefore, one of the objectives of the present invention is to provide a polynucleotide that is able to effectively induce the HCV infection. The polynucleotide comprises a modified HCV genome being capable of effectively inducing infection which comprises at least one alteration in a nucleotide sequence selected from the group consisting of a nucleotide sequence encoding E2 protein and a nucleotide sequence encoding p7 protein in the RNA genome of a JFH1 strain shown in SEQ ID NO: 1.

Another objective of the present invention is to provide a polynucleotide including a modified HCV recombinant genome that further includes a reporter gene and the HCV genome RNA.

Still another objective of the present invention is to provide a modified HCV containing a polynucleotide including a modified HCV recombinant genome or a polynucleotide including a modified HCV genome.

Still another objective of the present invention is to provide a vector containing a polynucleotide including a modified HCV recombinant genome or a polynucleotide comprising a modified HCV genome.

Still another objective of the present invention is to provide a transformant that is incorporated by a polynucleotide including a modified HCV recombinant genome or a polynucleotide including a modified HCV genome. It is preferred that the transformant can replicate the polynucleotide, producing virus particles, and infecting host cells.

Still another objective of the present invention is to provide virus particles of a modified HCV that contains a polynucleotide including a modified HCV recombinant genome or a polynucleotide including a modified HCV genome with mutation(s). It is also an objective of the present invention to provide virus particles of such modified HCVs that are obtained from the cell culture in which the transformant has been cultured.

It is also an objective of the present invention to provide HCV-infected cells using virus particles of the modified HCV according to the present invention. Another objective of the present invention is to provide a method for providing HCV infected-cells. The method includes the steps of culturing the transformant that is transfected with a modified HCV genome according to the present invention, obtaining virus particles from the cell culture in which the transformant has been cultured, and infecting other cells with the obtained virus particles.

Still another objective of the present invention is to provide an HCV vaccine or neutralizing antibody using virus particles as an antigen, in whole or in part, of the modified HCV according to the present invention.

Still another objective of the present invention is to provide a method for screening an anti-HCV substance or an HCV-therapeutic substance. The method can include the step of introducing into a host cell a polynucleotide including the modified HCV recombinant genome or a polynucleotide including a modified HCV genome with mutation(s), culturing the host cell in the presence of a given test substance, and assessing anti-HCV effects of the test substance. For the step of assessing anti-HCV effects of the test substance, one can use a method selected from the group consisting of observing whether the nucleotide sequence of the modified HCV or its virus particles are present, and quantifying virus infectivity. Alternatively, one can use a method selected from the group consisting of identifying reporter gene expression and quantifying the expression.

Another objective of the present invention is to provide a method for in vivo replication and/or expression of an extraneous gene. The method can include the step of inserting a RNA sequence coding for an extraneous gene into a polynucleotide including an HCV recombinant genome or into a polynucleotide including a modified HCV genome with mutation(s). The method can further include the step of transfecting a target cell with the polynucleotide as above in which the extraneous gene is inserted, such that the extraneous gene is replicated and expressed in the target cell.

Another objective of the present invention is to provide a vector to be used for replication of extraneous genes or for gene therapy. The vector is provided by using the modified HCV that contains a polynucleotide including a modified HCV genome.

### BRIEF DESCRIPTION OF THE DRAWINGS

FIG. 1 is a schematic view showing the structure of a JFH1 HCV construct according to one of the embodiments of the present invention, which include a reporter protein-coding region and cell-culture adaptive mutations.

FIG. 2 shows the results of a quantitative analysis by Western blotting using NS5a-antibodies and core-antibodies, which shows levels of expression of NS5a and core proteins, according to one of the embodiments of the present invention.

FIG. 3 is a graph identifying the levels of HCV RNAs in the transformants respectively transfected with the JFH 5a-GFP, JFH 5a-Rluc, JFH, and JFH pol⁻ viruses, according to one of the embodiments of the present invention.

FIG. 4 is a graph showing the activities of luciferase, as measured by the lapse of time in the transformants respectively transfected with the JFH 5a-Rluc and JFH pol⁻ viruses, according to one of the embodiments of the present invention.

FIG. 5 is a graph showing the expression of core protein and 5a-GFP protein, obtained by using fluorescence microscopy and core protein-antibodies, in the transformants respectively transfected with the JFH 5a-GFP, JFH 5a-Rluc, JFH, and JFH pol⁻ viruses, according to one of the embodiments of the present invention.

FIG. 6 shows results of the effects of IFN-α treatment, as verified by using transformants transfected with the JFH 5a-Rluc virus, according to one of the embodiments of the present invention.

FIG. 7 shows results of the effects of Ribavirin treatment, as verified by using the transformant transfected with the JFH 5a-Rluc virus, according to one of the embodiments of the present invention.

FIG. 8 shows results of the effects of BILN 2061 treatment, as verified by using the transformant transfected with the JFH 5a-Rluc virus, according to one of the embodiments of the present invention.

FIG. 9 is an image showing the changes in the transformant, in the absence of IFN-α, which was transfected with the JFH 5a-GFP RNA. The image was taken with a time-lapse confocal laser microscope every 12 hours for a total of 60 hours, according to one of the embodiments of the present invention.

FIG. 10 is a graph showing changing fluorescent levels indicated in absolute values in 8 transformants with no IFN-α treatment, which were transfected with the JFH 5a-GFP RNA, according to one of the embodiments of the present invention.

FIG. 11 is a graph showing changing fluorescent levels over time in 8 transformants in relative values against a starting value, i.e., a value given to the transformants with no IFN-α treatment, according to one of the embodiments of the present invention.

FIG. 12 is a graph showing averages indicated in relative values of fluorescent levels changing over time in the 8 transformants, in the absence of IFN-α, which were infected with the JFH 5a-GFP RNA, according to one of the embodiments of the present invention.

FIG. 13 is an image showing the changes on the transformants, in the presence of 1000 IU/ml of IFN-α, which were transfected with the JFH 5a-GFP RNA, according to one of the embodiments of the present invention. The image was taken with a time-lapse confocal laser microscope at every 12 hours for a total of 60 hours.

FIG. 14 is a graph showing changing fluorescent levels indicated in absolute values over time in 8 transformants, in the presence of 1000 μml of IFN-α, which were transfected with the JFH 5a-GFP RNA, according to one of the embodiments of the present invention.

FIG. 15 is a graph showing changing fluorescent levels indicated in relative values against a starting value over time in the 8 transformants, in the presence of 1000 IU/ml of IFN-α, which were transfected with the JFH 5a-GFP RNA, according to one of the embodiments of the present invention.

FIG. 16 is a graph showing the averages, by relative values, of fluorescent levels changing over time in the 8 transformants, in the presence of 1000 IU/ml of IFN-α, which were transfected with the JFH 5a-GFP RNA, according to one of the embodiments of the present invention.

FIG. 17 is a graph showing an infectivity comparison between the naive Huh 7.5.1 strain and selected strains, according to one of the embodiments of the present invention.

FIG. 18 is an image comparing infectivity between a cell-adapted virus and the original virus, the image obtained by examining core protein expression by using an immunocytochemistry method.

FIG. 19 is an image showing viral protein expression in cells infected with HCV that acquired cell-adaptive mutations and that were also capable of effectively replicating, to compare expression of core and NS5a-GFP proteins of the cell-adapted clones of Ad9, Ad12, and Ad16, according to one of the embodiments of the present invention.

FIG. 20 is an image showing the levels of NS5a-GFP protein expression, as indicated by the strength of florescence, of the cell-adapted clones of Ad9, Ad12, and Ad16, according to one of the embodiments of the present invention.

FIG. 21 is a graph showing the levels of TCID₅₀ in the cell-adapted clones of Ad9, Ad12, and Ad16, according to one of the embodiments of the present invention.

FIG. 22 shows results of the expression of core and NS5a-GFP proteins, according to one of the embodiments of the present invention.

FIG. 23 is a fluorescent image showing the NS5a-GFP protein expression in infected cells, according to one of the embodiments of the present invention.

FIG. 24 is a diagram showing the sites for restrictive enzymes and mutations in the cell-adapted clones of Ad9, Ad12, and Ad16, according to one of the embodiments of the present invention.

FIG. 25 is a diagram summarizing the alterations in nucleotide sequences in the cell-adapted clones of Ad9, Ad12, and Ad16, according to one of the embodiments of the present invention.

FIG. 26 is an image showing the results of an experiment that identified critical base changes contributing to enhanced virus production, among the changes found in the cell-adapted clone of Ad9, according to one of the embodiments of the present invention.

### DETAILED DESCRIPTION OF THE PREFERRED EMBODIMENTS

The present invention is explained in detail as follows.

As used herein, HCV refers to a positive-sensitive RNA virus, having a single stranded RNA viral genome of approximately 9.6 kb in length. The genome contains a 5′ untranslated region (5′ UTR) and a 3′ untranslated region (3′ UTR), with one long open reading frame (ORF) flanking the NTRs. Individual mature HCV proteins are produced by proteolytic processing of the precursor polypeptide encoded from the open reading frame. This proteolysis is catalyzed by a combination of both cellularly- and virally-encoded proteases, producing at least ten individual proteins. Those ten proteins consist of structural proteins, including core, E1, E2, and p7, and nonstructural proteins, including NS2, NS3, NS4a, NS4b, NS5a, and NS5b.

Also, as used herein, the term “modified HCV” refers to an HCV in which one or more of its naturally occurring sequences in its genome RNA is substituted and/or deleted, or an HCV in which an extraneous polynucleotide or gene is inserted into its genome RNA.

As used herein, the term “chimeric HCV genome RNA” refers to a combination of two genome RNAs coming from two different kinds of HCV.

As used herein, the term “HCV recombinant genome” refers to an HCV genome RNA that autonomously replicates, in which at least one extraneous polynucleotide is inserted into the naturally occurring HCV genome RNA. Alternatively, it can refer to an HCV genome RNA that autonomously replicates, in which at least one extraneous polynucleotide is inserted into the naturally occurring HCV genome RNA while at least one sequence in the naturally occurring HCV genome RNA was substituted or deleted.

As used herein, the term “reporter gene” refers to gene coding for a protein that is susceptible to quantitative analysis when expressed. Any reporter proteins known so far are applicable for the present invention, including but not limited to genes for Renilla luciferase, green fluorescent protein, firefly luciferase, red fluorescence protein, and secreted alkaline phosphatase (SeAP). It is preferred that more than one reporter gene is selected for the present invention from the group consisting of Renilla luciferase and green fluorescent protein.

Assays known to those skilled in the art for the detection and quantification of reporter genes are applicable for the present invention. In the case of using green fluorescent protein, for example, the level of protein expression is identifiable by observing green fluorescent protein that appears when cells are exposed to UV-irradiation. It is thus possible to observe the expression from living cells.

The present invention relates a polynucleotide including a modified HCV genome including at least one alteration in the protein coding nucleotide sequence(s) that encode one or more proteins selected from the group consisting of E2 and p7 proteins in the RNA genome of a JFH1 strain shown in SEQ ID NO: 1. SEQ ID NO: 1 is the genomic RNA of a JFH1 strain with no heterologous gene.

As the inventors of the present inventions found, since RNA replication and transfection of HCV can be effectively induced by substituting one or more nucleotide sequences encoded for at least one protein selected from the group consisting of E2 protein and p7 protein from the JFH1 strain of HCV, a polynucleotide with such modified HCV genome (or a modified HCV containing the polynucleotide) can provide a system for developing new anti-HCV agents.

The modified genome that effectively induces HCV infection can be from a cell-adaptive mutant of a JFH1 strain that produces the virus at a high rate, and the mutant virus, compared with the original HCV JFH1 strain, can produce viruses two to 100 times more effectively.

The E2 protein coding region to be altered on the JFH 1 genome RNA shown in SEQ ID NO: 1 can be nucleotide sequences 2027-2029 (corresponding to nucleotide sequences 1687-1689 of SEQ ID NO: 1). A preferred nucleotide to be altered is 2028 (corresponding to nucleotide 1688 of SEQ ID NO: 13). The alteration of the E2 protein encoding region can be made by at least one substituted nucleotide in the region of nucleotides of 2027-2029 of SEQ ID NO: 1 (corresponding to nucleotide sequences of 1687-1689 of SEQ ID NO: 13), the substituted nucleotide sequences not resulting in Threonine. Preferably, where at least one of the nucleotide sequences of 2027-2029 of SEQ ID NO: 1 (corresponding to nucleotides 1687-1689 of SEQ ID NO: 13) is substituted with another nucleotide sequence(s), the substituted nucleotide sequence(s) of 2027-2029 of SEQ ID NO: 1 (corresponding to nucleotides 1687-1689 of SEQ ID NO: 13) result in an altered E2 protein encoding region that codes for an amino acid selected from the group consisting of isoleucine, leucine, valine, phenylalanine, methionine, cysteine, alanine, glycine, proline, serine, tyrosine, tryptophan, glutamine, asparagine, histidine, glutamine acid, asparagine acid, lysine, and arginine. More preferably, the altered nucleotide sequence(s) of 2027-2029 of SEQ ID NO: 1 (corresponding to nucleotides 1687-1689 of SEQ ID NO: 13) results in an amino acid change from threonine to isoleucine.

In addition, a modification of a nucleotide sequence in the E2 protein encoding region can occur by altering the base A at the nucleotide sequence 2027 (corresponding to nucleotide sequence 1687 of SEQ ID NO: 13) to a base selected from the group consisting of U, T, G, and C. Another substitution of a nucleotide sequence in the E2 protein encoding region can occur by altering the base C at nucleotide sequence 2028 (corresponding to nucleotide sequence 1688 of SEQ ID NO: 13) to a base selected from the group consisting of U, T, G, and A. Still another substitution of a nucleotide in the E2 protein encoding region can occur by altering the base C at nucleotide sequence 2029 (corresponding to nucleotide sequence 1689 of SEQ ID NO: 13) to a base selected from the group consisting of U, T, G, and A. A preferred substitution of a nucleotide sequence in the E2 protein encoding region can occur by altering the base C at nucleotide sequence 2028 (corresponding to nucleotide sequence 1688 of SEQ ID NO: 13) to a base selected from the group consisting of U, T, G, and A. A more preferred substitution of a nucleotide sequence in the E2 protein encoding region can occur by altering the base C at nucleotide sequence 2028 (corresponding to nucleotide sequence 1688 of SEQ ID NO: 13) to U or T.

Alteration of p7 protein encoding region in the genome RNA of a JFH1 strain (as shown in SEQ ID. No: 1) can be at nucleotide sequences 2633-2635 of SEQ ID NO: 1 (corresponding to nucleotide sequences 2293-2295 of SEQ ID NO: 13). A preferred nucleotide sequence to be altered is nucleotide sequence 2633 (nucleotide sequence 2293 of SEQ ID NO: 13). The modified p7 protein encoding region can be made by at least one substituted nucleotide sequence in the region of nucleotide sequences 2633-2635 of SEQ ID NO: 1 (corresponding to nucleotides 2293-2295 of SEQ ID NO: 13), the substituted nucleotide sequence(s) not resulting in Asparagine. Preferably, where at least one nucleotide sequences among 2633-2635 of SEQ ID NO: 1 (corresponding to nucleotides 2293-2295 of SEQ ID NO: 13) is substituted for another nucleotide sequence(s), the substituted nucleotide sequence(s) of 2633-2635 of SEQ ID NO: 1 (corresponding to nucleotides 2293-2295 of SEQ ID NO: 13) result in the modified p7 protein-encoding region that codes for a protein selected from the group consisting of isoleucine, leucine, valine, phenylalanine, methionine, cysteine, alanine, glycine, proline, serine, tyrosine, tryptophan, glutamine, asparagine, histidine, glutamine acid, asparagines acid, lysine, and arginine. More preferably, the modified nucleotide sequence(s) of 2633-2635 of SEQ ID NO: 1 (corresponding to nucleotides 2293-2295 of SEQ ID NO: 13) results in an amino acid change to asparagine.

In addition, a substitution of a nucleotide in the p7 protein encoding region can occur by altering the base A at the nucleotide sequence 2633 (corresponding to nucleotide sequence 2293 of SEQ ID NO: 13) to a base selected from the group consisting of U, T, G, and C. Another substitution of a nucleotide in the p7 protein encoding region can occur by altering the base A at the nucleotide 263 (corresponding to nucleotide 2294 of SEQ ID NO: 13) to a base selected from the group consisting of U, T, G, and C. Still another substitution of a nucleotide in the p7 protein encoding region can occur by altering the base C at the nucleotide 2635 (corresponding to nucleotide sequence 2295 of SEQ ID NO: 13) to a base selected from the group consisting of U, T, G, and A. A preferred substitution of a nucleotide sequence in the p7 protein encoding region can occur by altering the base A at the nucleotide sequence 2633 (corresponding to nucleotide sequence 1688 of SEQ ID NO: 13) to a base selected from the group consisting of U, T, G, and C. A more preferred substitution of a nucleotide sequence in the p7 protein encoding region can occur by altering the base A at the nucleotide sequence 2633 (corresponding to nucleotide sequence 2293 of SEQ ID NO: 13) to G.

A polynucleotide including a modified genome that effectively induces HCV infection can be a polynucleotide in which a reporter gene is additionally included in the JFH1 genome RNA, in addition to the NS5a protein coding region.

At least one reporter gene can be selected from the group consisting of the genes for Renilla luciferase, green fluorescene protein, firefly luciferase, red fluorescence protein, and secreted alkaline phosphatase (SeAP). It is preferred that at least one reporter gene is selected from the group consisting of the genes for Renilla luciferase and green fluorescene protein.

More specifically, in a modified HCV including at least one alteration in the protein coding nucleotide sequence(s) that encodes one or more proteins selected from the group consisting of E2 and p7 proteins in the RNA genome of a JFH1 strain shown in SEQ ID NO: 1, a reporter gene is inserted into the NS5a protein coding sequence, and preferably, to the C-terminal region of the NS5a-coding sequence. More preferably, the reporter gene is inserted right after at least one nucleotide selected from the group consisting of 7176, 7179, 7182, 7185 and 7188th nucleotides in the genome RNA of the JFH1 strain represented by SEQ ID NO:1 (corresponding to 6836, 6839, 6842, 6845 and 6848th nucleotides of SEQ ID: 13).

Still more preferably, the reporter gene can be incorporated between the nucleotides of 6842 and 6843 in the genomic RNA of the JFH1 strain represented by SEQ ID NO:1 (i.e., between the region coding for amino acid 418 of NS5a and the region coding for 419th amino acid of NS5a).

The present invention also relates to the cDNA of the modified HCV or the genome RNA of the modified HCV, the vector being able to effectively induce infection.

The present invention also relates to a polynucleotide containing a modified HCV recombinant genome in which a reporter gene and the HCV genome are included.

The inventors of the present invention have found that a reporter gene inserted in the NS5a-coding region of JFH1 strain has little effect on viral activities regarding HCV life cycle, replication, and infection of HCV. Thus it is possible to have reporter protein expressed without addition of heterologous controlling element such as internal ribosome entry site (IRES) of encephalomyocarditis virus (EMCV).

The HCV containing reporter gene provides a system for studying HCV's life cycle and developing anti-HCV agents. The HCV genome RNA possesses autonomous replicative competence. Preferably, the HCV genome RNA can be the genome RNA of the JFH1 strain (SEQ ID No:1), chimeric HCV genome RNA, a mutant genome RNA having an alteration in the nucleotide sequence coding for a protein selected from the group consisting of E2 protein and p7protein of the JFH1 strain (SEQ ID No:1), or a modified HCV recombinant RNA. The more preferred HCV genome RNA is a mutant genome RNA having an alteration in the nucleotide sequence coding for a protein selected from the group consisting of E2 protein and p7protein of the JFH1 strain (SEQ ID No:1).

Specifically, a recombinant genome RNA can be an HCV genome RNA that has autonomous replication competence and is able to infect host cells, preferably an HCV genome RNA in which at least one nucleotide sequence on the genome RNA of JFH 1 strain (SEQ ID No:1) is substituted and/or deleted while having at least one heterologous polynucleotide.

As another embodiment of the present invention, the chimeric HCV can include the nucleotide sequences from the HCV genome RNA—which are the 5′-untranslated region, core protein-encoding sequence, E1 protein-encoding sequence, E2 protein-encoding sequence, p7 protein-encoding sequence, and NS2 protein-encoding sequence—and the nucleotide sequences from the JFH1 strain (SEQ ID NO:1)—which are NS3 protein-encoding sequence, NS4a protein-encoding sequence, NS4b protein-encoding sequence, NS5a protein-encoding sequence, NS5b protein-encoding sequence, and the 3′-untransalated region.

The present invention relates to a vector containing a polynucleotide including a modified HCV genome wherein the infection-inducing JFH1 genome is modified at the nucleotide sequence(s) for one or more proteins selected from the group consisting of E2 and p7 proteins of the JFH1 strain of HCV (the SEQ ID NO: 1); and alternatively the vector can contain a polynucleotide including a modified HCV recombinant genome wherein a reporter gene and the HCV genome RNA are included. The vectors can be used for the expression of heterologous proteins or for gene therapy.

Since the modified HCVs or the modified HCV virus particles from the same are hepatocyte-targeting, the resulting vector can be a hepatocyte-targeting virus or a virus vector for hepatocytes. The hepatocyte-targeting virus or a virus vector for hepatocytes can be used to infect host cells with the modified HCVs for the purpose of performing HCV related studies. For gene therapy, a virus to be used can be a modified HCV that is unable to do self-infection, meaning that the virus is able to infect only when provided with viral gene products from other viruses or the host cell, and is unable to self-proliferate.

In addition, the present invention relates to a transformant incorporating a polynucleotide including a modified HCV recombinant genome wherein a reporter gene and the HCV genome RNA are included; and alternatively the transformant may be one that has incorporated a polynucleotide including a modified HCV genome in which the effectively infection-inducing JFH1 genome is modified at the nucleotide sequence(s) for one or more proteins selected from the group consisting of E2 and p7 proteins of the JFH1 strain of HIV (the SEQ ID NO: 1).

The transformant can be a host cell that contains a polynucleotide including the modified HCV recombinant genome RNA or the modified HCV genome RNA, the host cell supporting replication of the modified HCV recombinant genome RNA or the modified HCV genome and also generating virus particles thereof.

Although any cell that is susceptible to subculture can be used as a host cell, eukaryotic, and more preferably human cells, are preferred. Preferably, human cells to be used as a host cell include the cell lines of human kidney origin, human cervix origin, or human embryonic kidney origin. It is preferred that the host cells are proliferative like tumor cell lines and hepatocyte cell lines, more preferably, Huh 7, HepG2, IMY-N9, HeLa, or 293 cells. These cells are commercially available, or may be obtained from cell banks. Alternatively, a researcher may obtain cells from, for example, tumor cells or hepatocyte cells, by using chemotaxis.

Huh 7 cells to be used can be the Huh 7.5.1 cell line, which is known to present higher permissiveness (Zhong et al., Proc. Natl. Acad. Sci. USA, 102, 9294-9299), or can be the Huh 7.5.9 cells, which has shown to have high permissiveness and has been newly named so in an embodiment according to the present invention.

Transformation of the host cell (i.e., by transfection of the host cell with the polynucleotide, the modified HCV recombinant HCV genomic RNA, or the modified HCV genome RNA) can be achieved via a known method, for example, a method of packaging the polynucleotide in a virus and then introducing the virus into the host cell, or a method of directly introducing the polynucleotide into the cell (direct uptake). Specifically, the transformation (transfection) can be performed via electroporation, particle bombardment, lipofection, microinjection, or DEAE sepharose, although the electroporation is preferred.

One can tell whether transformation has successfully been achieved or whether the modified HCV recombinant genomic RNA or the modified HCV genomic RNA is replicable by performing a known RNA extraction method. In order to know whether HCV proteins are found in the protein collected from the host cell, one may use a known protein extraction method, preferably by examining whether a report gene was expressed or by quantifying protein expression.

HCV infected cells, which were infected by transfection or with the virus particles from the transformant, can be used as a system for screening pro- or anti-agents regarding HCV replication, reconstruction of virus particles, and discharge of virus particles.

One of the advantages of using the transformant according to the present invention is that a researcher is able to easily identify the introduction or replication of the modified HCV recombinant RNA or the modified HCV genomic RNA, by observing reporter protein expression and its intensity. Another advantage is that the polynucleotide of modified HCV recombinant RNA or the polynucleotide of the modified HCV genomic RNA replicate efficiently.

The advantages described as above lead to various uses available to the transformant according to the present invention, which will be described below.

One aspect of the present invention relates a method for manufacturing RNA including an HCV genome sequence, the method including the steps of culturing the transformant, extracting RNAs from the cell culture of the transformant, isolating the HCV genome RNA from the extracted RNAs, and isolating and purifying the HCV genome RNA. Since the RNA resulting from the above contains an HCV genome sequence, a researcher is able to do a more precise analysis regarding an HCV genome.

Another aspect of the present invention is that the transformant according to the present invention can be used to manufacture HCV proteins. A method used to manufacture the HCV proteins can be a known one, for example a classical method including the steps of culturing the transformant and extracting proteins from the cell culture of the transformant.

Another aspect of the present invention is that the transformant according to the present invention can be used as a system for the screening of pro- or anti-agents for HCV infection of the host cell. Specifically, the system includes the steps of culturing the transformant in the presence of a given test substance, extracting HCV genome RNA, virus particles, or reporter protein from the cell culture, and verifying whether the replication of HCV genome RNA or formation of virus particles was facilitated or inhibited in the presence of the test substance.

The extraction of HCV genome RNA, virus particles, or reporter protein can be performed by the methods described previously or by the methods that will be subsequently described in examples to follow. The system can be used to manufacture or test prophylactic, therapeutic, and diagnostic agents.

Specifically, some examples of using the present invention as a test system are provided below.

(1) Exploration of anti-viral agents that inhibit the proliferation and infection of HCV.

Such anti-viral agents can include organic compounds that directly or indirectly affect the proliferation and infectivity of HCV, or alternatively can be antisense oligonucleotides resulting in hybridization with an HCV genome or its complimentary strand, thereby directly or indirectly affecting the proliferation or gene expression of HCV.

(2) Assessment of various anti-viral materials during cell culture.

Such anti-viral materials can be obtained through, for example, rational drug design or high throughput screening (e.g. purified enzymes).

(3) Identification of HCV's new targets in the host cell, for the treatment of HCV infected patients.

For example, it is possible to use HCV genome RNA-replicating cells according to the present invention, to identify host cell proteins that serve an important role for the HCV proliferation.

(4) Assessment of HCV's acquired resistance to anti-HCV agents and identification of the mutations conferring the resistance.

(5) Manufacturing virus proteins that will be used as an antigen for developing, producing, and assessing prophylactic and therapeutic treatments for HCV infection.

(6) Manufacturing an attenuated HCV or virus proteins, in order to use them as an antigen for developing, producing, and assessing vaccines for HCV infection.

(7) Gene therapy using an HCV as a vector

The present invention also relates to virus particles of a modified HCV. The virus particles of a modified HCV can be the product of the modified HCV in which the effectively infection-inducing JFH1 genome is modified at the nucleotide sequence(s) coding for one or more proteins, the proteins being selected from the group consisting of E2 and p7 proteins of the JFH1 strain of HIV (SEQ ID NO: 1).

The virus particles of the modified HCV can be obtained from the cell culture of the transformant according to the present invention.

The cell culture used in the above is a culture fluid in which the transformant is incubated, and can be a cell suspension or a cell free supernatant.

The transformant (i.e., a cell transformed by incorporating a polynucleotide, modified HCV recombinant RNA, or a modified HCV genome RNA, according to the present invention, into the host cell) is able to generate HCV virus particles in vitro. In other words, one can easily obtain HCV particles by growing the transformant in the culture medium and then collecting virus particles from the cell culture (preferably, culture supernatant). The virus particles released into the cell culture demonstrate infectivity to a cell, preferably to an HCV susceptible cell.

In addition, the present invention relates to an HCV-infected cell that is infected by virus particles of the modified HCV according to the present invention.

The HCV-infected cell is characterized by the infection by the modified HCV according to the present invention, the modified HCV containing a polynucleotide including the modified HCV recombinant genome in which a reporter gene and the HCV genome RNA or a polynucleotide including the modified HCV genome in which the effectively infection-inducing JFH1 genome is modified at the nucleotide sequence(s) for one or more proteins selected from the group consisting of E2 and p7 proteins of the JFH1 strain of HIV (SEQ ID NO: 1).

The present invention also relates to a method for manufacturing an HCV-infected cell, the method including the steps of culturing the transformant, and infecting a target cell (preferably a host cell, and more preferably an HCV sensitive cell) with the cell culture or virus particles of the transformant.

HCV-permissive cells are those that are permissive to HCV infection, and for the present invention, the HCV-permissive cell to be used can come from, without being limited to, the lines of hepatocytes or lymphoid cells. Specifically, the hepatocyte cells, for example, can be primary-cultured liver cells, Huh 7, HepG1, IMY-N9, HeLa, or 293 cells.

Once a cell (for example an HCV-permissive cell) is infected with the HCV virus particles, the cell supports replication of the modified HCV genome RNA or a polynucleotide thereof, or produce virus particles. In other words, by infecting cells with the virus particles produced from the transformant according to the present invention, the modified HCV genome RNA or a polynucleotide thereof can be replicated in the infected cell, thereby allowing one to manufacture the virus particles in a large amount.

By infecting animals like chimpanzees with the HCV virus particles, it is possible to cause HCV-originated disease, such as hepatitis, in the animal.

The present invention also relates to an HCV vaccine or attenuated antigen that can be developed by using the modified HCV according to the present invention as an antigen, in whole or in part.

The present invention also relates to a method for preparing a vaccine, the method using the HCV virus particle as an antigen, in whole or in part, according to the present invention, or a particle made of the HCV's outer shell that is reconstructed to change the targeting of the virus, in whole or in part.

By using the HCV virus particle as an antigen, in whole or in part, or the particle made of the HCV outer shell that is reconstructed to change the targeting of the virus, in whole or in part, one may also prepare an attenuated antibody.

The present invention also relates to a method of gene therapy, the therapy using a certain product according to the present invention, that is, the polynucleotide including a modified HCV according to the present invention, the virus particle of the modified HCV, in whole or in part. For the method of gene therapy according to the present invention, a known method can be used that utilizes viral genomic RNA or a part thereof.

The present invention also relates to a method for screening anti-HCV material, the method including the step of cultivating a host cell that has been transfected in the presence of a given test substance with a polynucleotide including a modified HCV recombinant genome wherein a reporter gene and the HCV genome RNA are included; and alternatively, the transfection can be done by a polynucleotide including a modified HCV genome that is effectively infection-inducing, wherein the modified HCV genome including alterations in the sequence(s) encoding one or more proteins selected from the group consisting of E2 and p7 proteins of a JFH1 strain shown in SEQ ID NO: 1. The method further includes the step of assessing the anti-HCV effect of the test substance.

To further illustrate, in the presence of the test substance, a modified HCV genome RNA having a reporter gene or the genome RNA of a mutant of the HCV JFH1 strain is introduced into a host cell, subsequently resulting in the replication of the HCV genomic RNA. Then the host cell transfected (transformant) is cultured. The HCV genomic RNA or the HCV virus particles are extracted from the transformant cell culture. By examining whether the replication of the replicon RNA or the HCV genomic RNA was facilitated or inhibited or whether the formation or release of virus particles was facilitated or inhibited, one can screen a substance that facilitates or inhibits viral activities of HCV. As to the HCV genome RNA extracted from the cell culture, it is preferred to measure the amount or existence of the HCV genome RNA in the total RNA extracted, or the ratio of the HCV genomic to the total RNAs. As to the virus particles extracted from the cell culture (preferably culture supernatants), one may measure the proportion, amount, or existence of the HCV proteins in the cell culture or measure the amount of protein expressed from the reporter gene.

Anti-HCV effects of a test substance include effects of inhibiting HCV activities, inhibiting HCV infection, inhibiting the replication of the HCV genome RNA, and inhibiting expression of HCV proteins.

For the step of assessing the anti-HCV effect of a test substance, one may choose at least one method selected from the group consisting of methods of detecting the presence of nucleotides of the modified HCV or the virus particle and of quantifying the activities thereof. Alternatively, one may choose at least one method selected from the group consisting of methods of detecting the presence of a reporter protein expressed and of quantifying the expression.

For example, when a polynucleotide including the gene for Renilla luciferase or green fluorescence protein is used as a reporter gene, anti-HCV effects of the test substance can be assessable by measuring the degree of luciferase activity or by quantitatively measuring fluorescence protein (i.e. measuring fluorescence intensities).

One can assess anti-HCV effects of a given test substance by observing whether the amount of a modified HCV or virus particles produced has decreased, or whether the expression of the reporter gene has reduced when the test substance is applied or with increased concentrations of the test substance applied.

In addition, one quantitatively measures anti-HCV effects of anti-HCV substances in individual cells and can therefore screen for them, by using a polynucleotide including a reporter gene.

The present invention also relates to a method for quantifying HCV infectivity, the method including the step of introducing into host cells a polynucleotide including a modified HCV genome that is effectively infection-inducing, wherein the modified HCV genome including alterations in the sequence(s) encoding one or more proteins selected from the group consisting of E2 and p7 proteins of a JFH1 strain shown in SEQ ID NO: 1, and the step of quantitatively measuring HCV infectivity.

For the step of quantifying HCV infectivity, one can measure the amount of the polynucleotide including the modified HCVs, or the modified HCV recombinant genome, the modified HCV with mutations, according to the present invention. Alternatively, the amount of protein expression of reporter gene can be measured.

A standard procedure to quantitatively measure nucleotides can be used for the polynucleotides. The protein expression can be quantitatively measured by quantitative analysis for the reporter protein expression or fluorescence intensities.

The present invention also relates to a method for identifying cells that are permissive to HCV infection. With the method one can identify a cell that incorporates inside a polynucleotide including a modified HCV recombinant genome wherein a reporter gene and the HCV genome RNA are included, then replicates the HCV genomic RNA, and eventually produces virus particles. The expression of the reporter protein can be quantitatively measured.

The present invention also relates a method for in vivo replication and/or in vivo expression of a heterologous gene. The method includes the step of inserting RNA sequence coding for a heterologous gene into a polynucleotide including a modified HCV recombinant genome wherein a reporter gene and the HCV genome RNA are included. Alternatively, a polynucleotide to which the heterologous gene is inserted can include a modified HCV genome including at least one alteration in the protein coding nucleotide sequence(s) that encodes one or more proteins selected from the group consisting of E2 and p7 proteins in the RNA genome of a JFH1 strain shown in SEQ ID NO: 1. The method further includes the step of introducing the polynucleotide into a target cell so as that the cell supports replication of viral RNA and expresses the polynucleotide.

With the present invention, one can incorporate a heterologous gene into a target cell and have the target cell support replication of HCV RNA and express the heterologous gene, by inserting the RNA coding sequence for the heterologous gene into an HCV genome RNA and then introducing the modified HCV into the target cell.

After replacing the E1 protein coding sequence and/or the E2 protein coding sequence in the HCV genome RNA with RNA sequences coding for the outer shell (envelope) of viruses originated from other species, one can introduce the resulting RNA into a cell and have the cell produce virus particles. In this manner one can create RNA having infectivity with various species. As a variation of the method described above, a heterologous gene can be inserted into the HCV genome RNA. In this variation, the heterologous gene can be expressed in various cells thanks to the modified HCV according to the present invention, whose targeting is engineered to recognize certain cells as intended.

The present invention also relates to a method for producing a virus vector containing a heterologous gene. The method includes the step of inserting the RNA sequence for a heterologous gene. The method further includes the step of creating a transformant by transfecting a host cell with the HCV genome RNA having the heterologous gene. The method further includes the step of producing virus particles by culturing the transformant.

The present invention will be described more specifically based on the following examples and drawings. However, the technical scope of the present invention is not limited to these examples.

### Example 1

### Cloning of the JFH 5a-GFP and JFH 5a-Rluc Plasmids, which Produce Reporter Proteins

1-1: Cloning of the JFH 5a-PmeI Plasmid

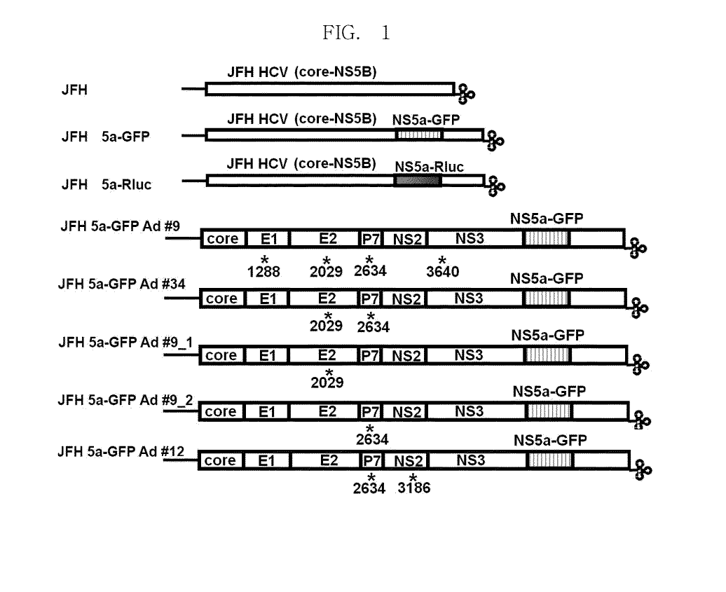

To express a reporter protein at the NS5a region in the known JFH construct as indicated in FIG. 1, particularly, to express reporter protein between the 2394^{th} and 2395^{th} amino acid coding sequences (418^{th} and 419^{th} amino acids in NS5a), a nucleotide sequence that can be cleaved by Pme I is inserted into the above-described region in the JFH 1 genome.

In particular, two DNAs were PCR-amplified using the two sets of primers in Table 1 and the JFH 1 plasmid (SEQ ID NO: 1) as a template.

TABLE 1

| | | SEQ |
|---|---|---|
| | | ID |
| Name | nucleotide sequence (5′→3′) | NO: |
| #1 forward primer | 5′-CCATCAAGACCTTTGGCC-3′ | 2 |
| #1 reverse primer | 5′-GAGGGGGTGTTTAAACAGGGGGGGCA | 3 |
| | TAGAGGAGGC-3′ | |
| #2 forward primer | 5-CTGTTTAAACACCCCCTCGAGGGGGAG | 4 |
| | CCTGG-3′ | |
| #2 reverse primer | 5′-TTGGCCATGATGGTTGTG-3′ | 5 |

The two kinds of DNAs amplified above were combined together through PCR. Subsequently, they were placed into the JFH 1 plasmid, with the use of the restriction enzymes Rsr II and Hpa I.

1-2: Cloning of JFH 5a-GFP and JFH 5a-Rluc Plasmids

The DNAs encoding Renilla luciferase and GFP were amplified using the primers in Table 2 below.

TABLE 2

| | | SEQ |
|---|---|---|
| | | ID |
| Name | Nucleotide sequence (5′→3′) | NO: |
| Rluc | 5′-ACTTACGTAACTTCGAAAGTTTATGATCC-3′ | 6 |
| forward | | |
| primer | | |
| Rluc | 5′-ACTGATATCTTGTTCATTTTTGAGAACTCGC-3′ | 7 |
| reverse | | |
| primer | | |
| GFP | 5′-ATCTACGTAGTGAGCAAGGGCGAGGAG-3′ | 8 |
| forward | | |
| primer | | |
| GFP | 5′-ATCGATATCCTTGTACAGCTCGTCCAT-3′ | 9 |
| reverse | | |
| primer | | |

The DNAs amplified from the above were treated with the restriction enzymes EcoR V and SnaB I to produce an insert, which in turn was inserted into the JFH 5a-PmeI plasmid of Example 1-1. Clones including the insert were screened. The clones including Rluc were named JFH 5a-Rluc, while the clones including GFP were named JFH 5a-GFP.

### Example 2

### Infection with JFH 5a-GFP and JFH 5a-Rluc Viruses

2-1: Synthesis of JFH 5a-GFP and JFH 5a-Rluc RNAs

RNAs were generated via in vitro transcription of the JFH 5a-GFP and JFH 5a-Rluc plasmids. Specifically, 16 μg of plasmids were treated with the restriction enzyme Xba I, and then single strands were treated with mung bean nuclease for removal. DNA templates were isolated by using phenol extraction and ethanol precipitation. The templates were transcribed into RNA by RNA polymerase (Stratagene Inc.) and were then isolated from the resulting RNAs by using DNase (Ambion Inc.). The RNA molecules were purified and collected by phenol extraction and ethanol precipitation and were dissolved in nuclease-free water. The RNAs were quantitatively measured by using a UV spectrophotometer and were run on a 1% agarose gel to observe whether the RNAs were generated as intended.

2-2: Preparation and Infection of JFH 5a-GFP and JFH 5a-Rluc viruses

The RNAs gained in Example 2-1 and the JFH pol-RNAs were compared with each other in terms of viral protein expression in an infected host cell, by introducing them into an Huh 7.5.1 cell line via electroporation. The JFH pol-RNA was used as a negative control because it contains a mutation at the catalytic site of the RNA polymerase NS5b (lane 2 on panels NS5a and core in FIG. 1B), and cannot replicate.

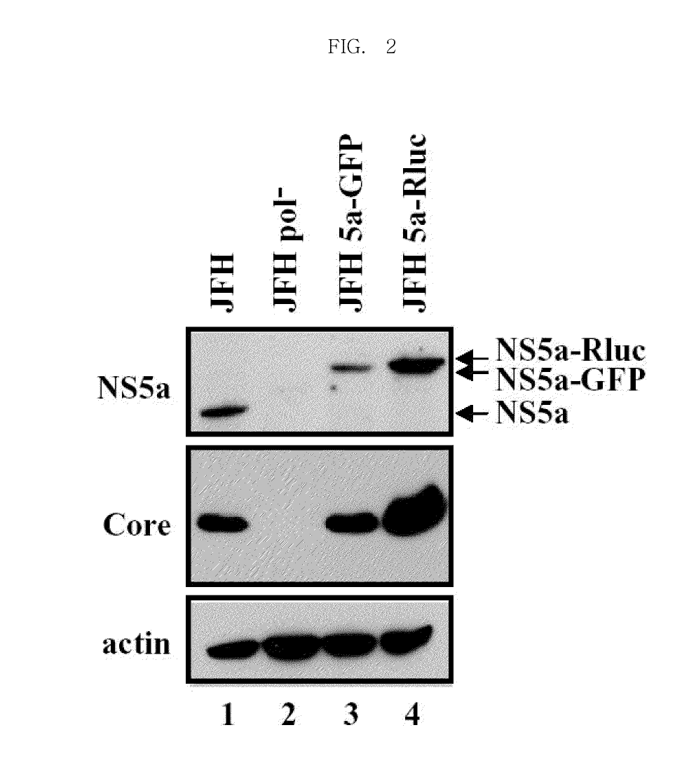

Three days after the transfection, cell lysates were prepared and the levels of the NS5a protein and core protein were assessed by Western-blot analysis using anti-NS5a and anti-core antibodies (Provided by Dr. Ralf Bartenschlager at University of Heidelberg). The results are indicated in FIG. 2.

As shown in FIG. 2, proteins accounting for Renilla luciferase and GFP were well expressed, and similar levels of core protein were expressed in the cells transfected with JFH and JFH 5a-GFP RNAs. Neither NS5a nor core protein was detected in the cells transfected with JFH pol-RNA.

2-3: Assessment of Luciferase Activities and Green Fluorescence in the JFH 5a-GFP and JFH 5a-Rluc Virus-Infected cells.

By transfecting RNAs in Example 2-1 (“modified RNA of Example 2-1”), the JFH RNAs, and the JFH pol-RNAs into Huh 7.5.1 cells, a transformant was obtained. Eight days after transfection, the transformant was removed to obtain 100 μl of cell-free supernatant. The above-obtained cell-free supernatant was then centrifuged and filtered through a 0.45 μm filter. The filtered culture was used to infect the Huh 7.5.1 cell line.

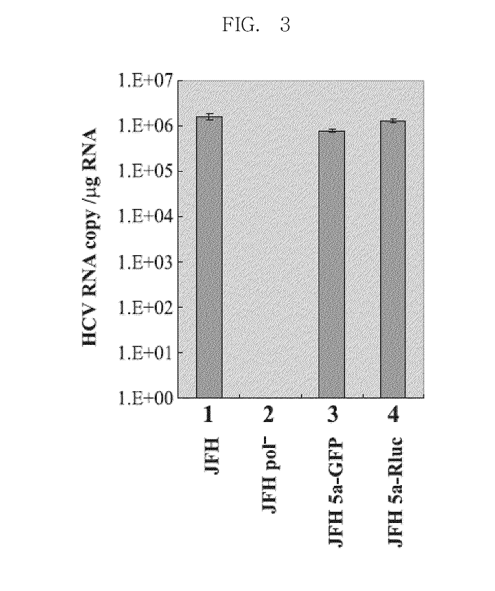

Three days after infection of the Huh 7.5.1 cells, total cellular RNA was isolated from infected cells and the level of HCV RNA was measured by quantitative reverse transcription PCR, specifically, real-time reverse transcription PCR (real-time RT PCR). GADPH (glyceraldehyde-3-phosphate dehydrogenase) mRNA was used as an internal RNA control, the result of which is indicated in FIG. 3.

Levels of HCV RNAs indicated in FIG. 3 were indicated by copy number per 1 μg of RNA. As FIG. 3 shows, similar levels of HCV RNAs were detected in cells infected with the JFH, JFH 5a-GFP, and JFH 5a-Rluc viruses. By contrast, HCV RNA was not detectable in cells infected with the culture supernatant obtained from cells transfected with the JFH pol-RNA

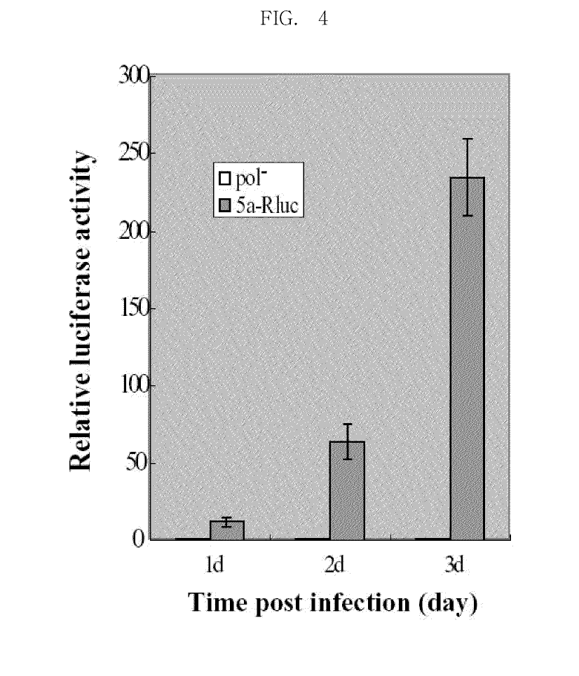

Cells infected with the culture supernatant obtained from the transformant transfected with the JFH 5a-Rluc RNA were measured for their luciferase activities. Measurement was performed three times respectively after 1, 2, and 3 days after infection. The results are shown in FIG. 4. Cells transfected with the JFH pol-RNA were again used as negative RNA control and measured for luciferase activity.

As shown in FIG. 4, no luciferase activity was detectable for the transformant transfected with the JFH pol-RNA, while luciferase activity increased over time for the transformant transfected with the JFH 5a-GFP. From the result of increased activity of luciferase over time, a finding is made that the transformant transfected with the JFH 5a-GFP provides a system for sensitive and quantitative assessment of viral infection.

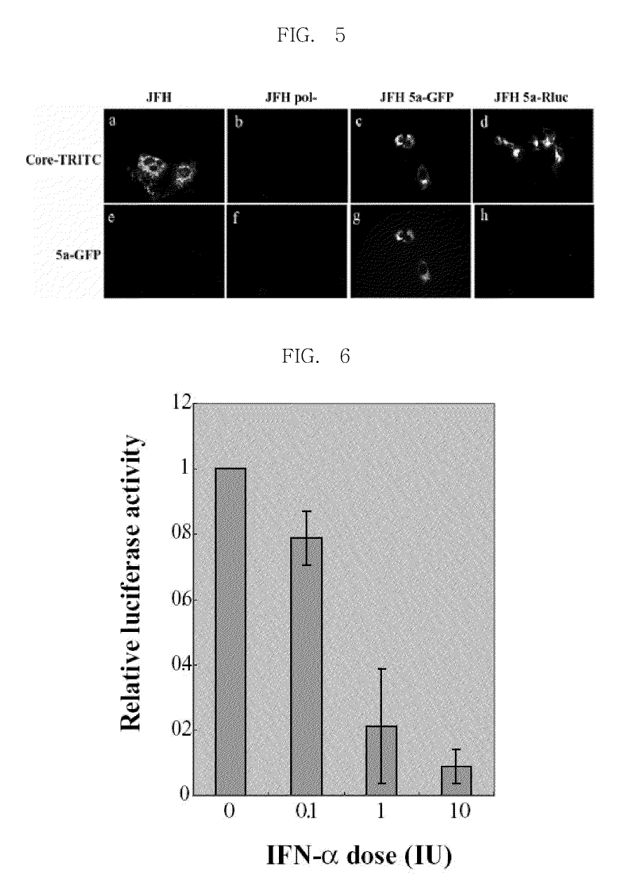

Infectivity of transformant transfected with the JFH 5a-GFP was also evaluated by fluorescence microscopy. Specifically, naïve Huh 7.5.1 cells were inoculated with culture supernatants of the transformant transfected with the modified RNA of Example 2-1, JFH RNA, and JFH pol-RNA. Infectivity was measured by an immunocytochemical method using an antibody against HCV core protein. The results are shown in FIG. 5.

FIG. 5 indicates that infection was readily detectable for the JFH, JFH 5a-GFP, and JFH 5a-Rluc viruses (panels a, c, and d in FIG. 5), whereas no core-expressing cells were found for inoculation with the JFH pol⁻ virus (panel b in FIG. 5). Moreover, in the same core-expressing cells, 5a-GFP fluorescence was observed only for the inoculation with the JFH 5a-GFP virus. Accordingly, it was found that one can identify and quantify virus infection by conveniently observing green fluorescence of 5a-GFP protein.

### Example 3

### Examination of the Anti-Viral Activities of Virus Inhibitors

Taking advantage of the present invention's ability to quantify the infection by the JFH 5a-Rluc virus, the inventors examined anti-viral activities of IFN-α, ribavirin, and BILN 2061, which is an NS3 protease inhibitor.

3-1: Examination of the Anti-Viral Activities of Virus Inhibitors

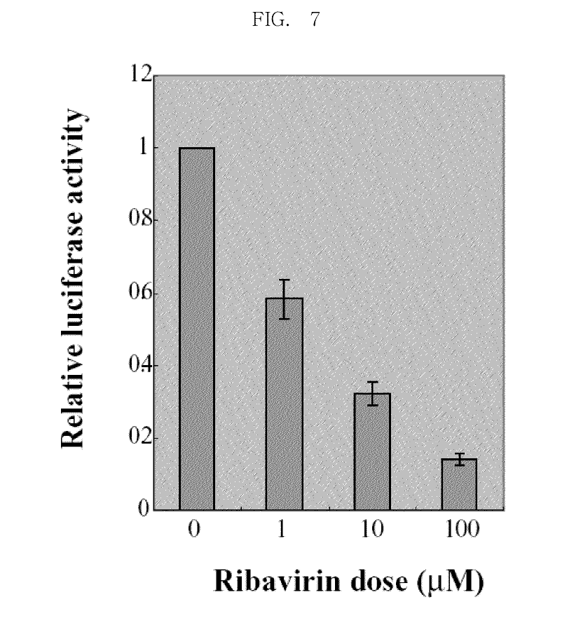

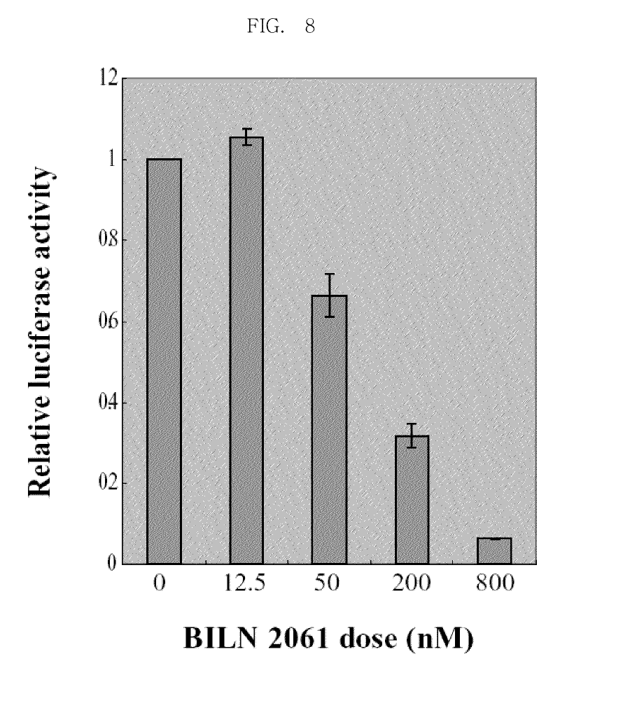

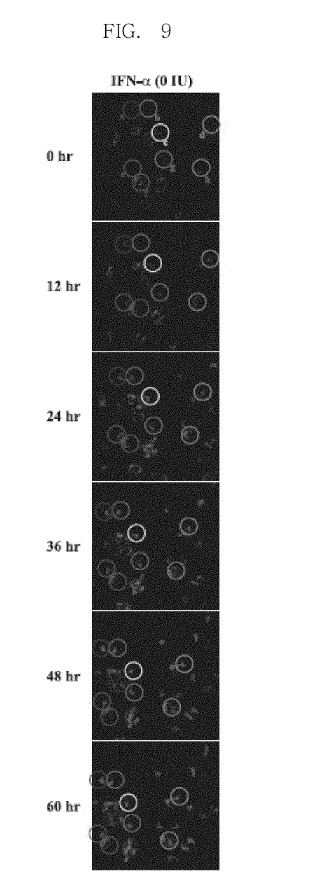

After infecting Huh 7.5.1 cells with culture supernatant obtained from the transformant transfected with the JFH 5a-Rluc RNA (the transformant was transformed with the introduction of JFH 5a-Rluc RNA via electroporation into Huh 7.5.1 cells, in the example 2-1), the amount of the anti-viral agents were maintained constantly throughout three days of culturing. The anti-viral agents used were IFN-α, ribavirin, and BILN 2061. After the three days, proliferation of JFH 5a-Rluc virus in the infected cells was tracked. FIGS. 6 to 8 show the results for the three anti-viral agents, respectively.

The results shown in the FIGS. 6 to 8 illustrate that luciferase activities vary in a dose-dependent manner, i.e., they vary depending on the concentration of the anti-viral agents applied. The values are presented as relative values, conferring a value of 1 on the case in which no anti-viral agent was applied. The median effective concentrations (EC50) of IFN-α and BILN 2061 against the JFH 5a-Rluc virus were similar to those against the J6/JFH virus, as previously reported by Lindenbach et al. This result indicates that the transformed JFH 5a-Rluc including the heterologous polypeptide responds to anti-viral agents in a similar manner as the normal JFH virus without such heterologous polypeptide and that the modified JFH 5a-Rluc virus possesses a similar life cycle as HCV's.

Since anti-viral effects in the modified virus JFH 5a-Rluc are similarly observed as in the HCV virus, the modified HCV having a reporter gene according to the present invention provides an effective system for exploring a new anti-viral agent.

3-2: Real Time Assessment of Anti-Viral Activity of IFN-α in Individual HCV-Infected Cells

Huh 7.5.1 cells were infected with culture supernatant obtained from the transformant transfected with JFH 5a-GFP RNA (the transformant was transformed with the introduction of JFH 5a-GFP RNA via electroporation into Huh 7.5.1 cells, in Example 2-1).

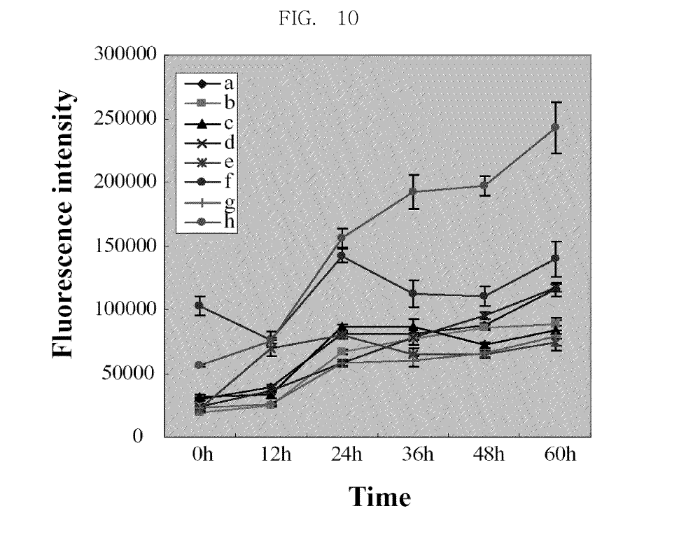

After treating or mock-treating (i.e., no IFN-α treated) the transformant with IFN-α, GFP fluorescence was monitored every 12 hours up to 60 hours by using time-lapse confocal microscopy (Zeiss LSM 5 Live). For time-lapse imaging, coverslips were mounted onto the microscope stage, which was equipped with a temperature- and gas-controlled chamber (Chamide IC, Live Cell Instrument, Korea). The comparative results between the cases of IFN-α treatment and mock-treatment are shown in FIGS. 9 and 13.

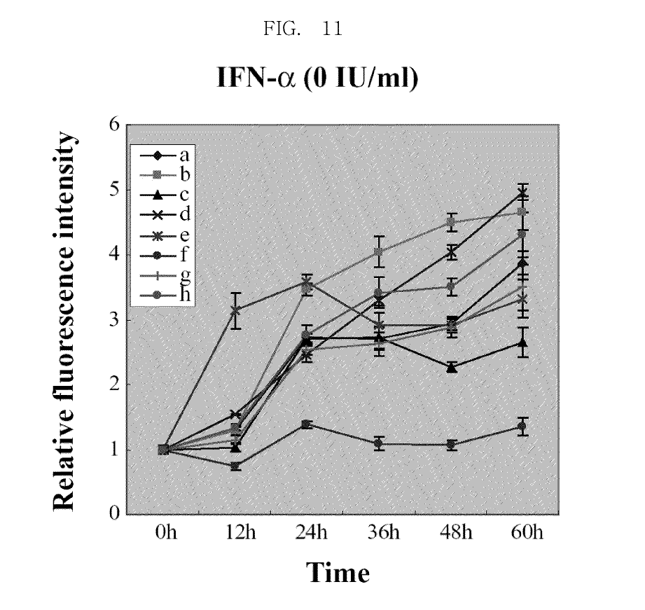

In addition, quantitative analyses of the fluorescence images of 8 cells were made by using MetaMorph software. The results of the analyses are shown in FIGS. 10-12, and FIGS. 14-16. The grouping of the figures was made depending on whether the cell was treated with IFN-α. The 8 cells were selected among those that demonstrated the strongest intensities.

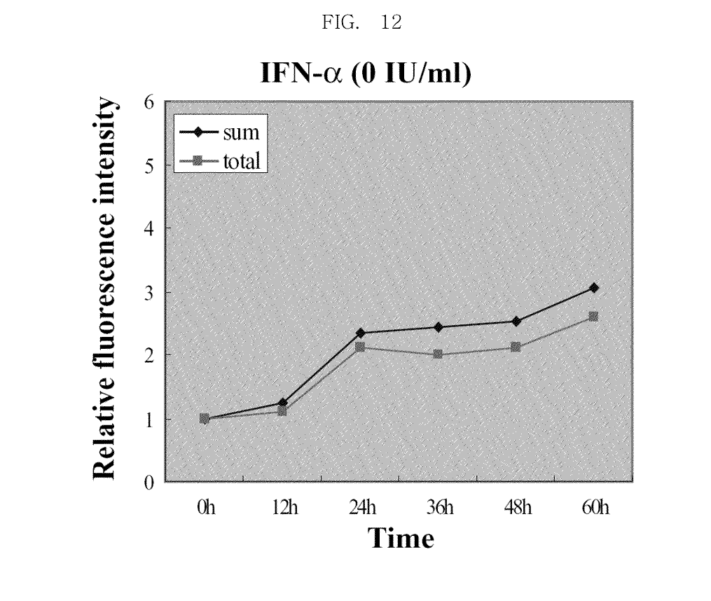

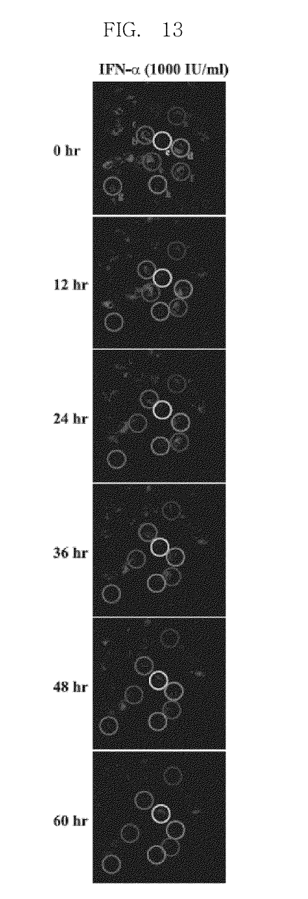

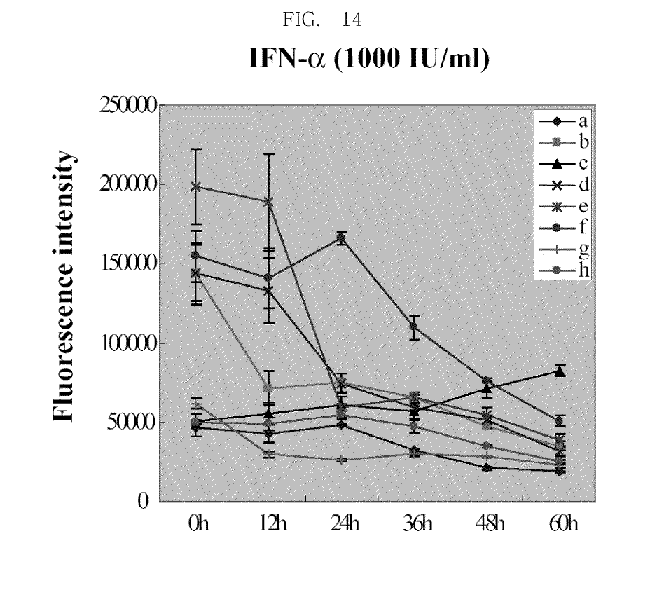

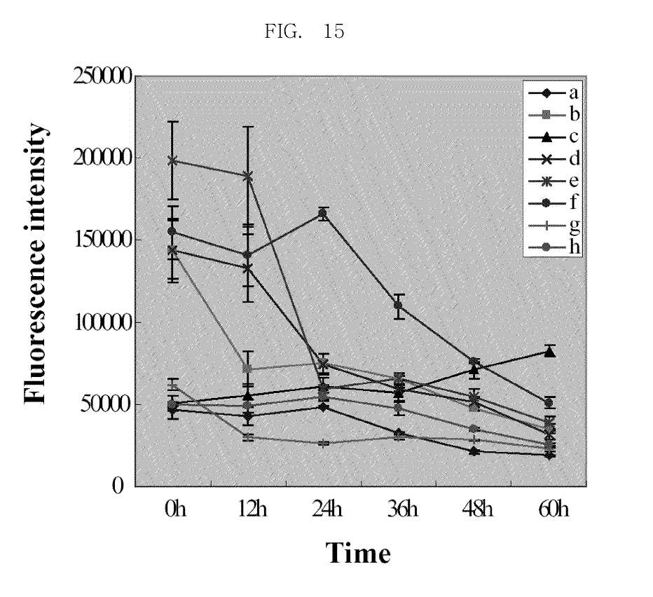

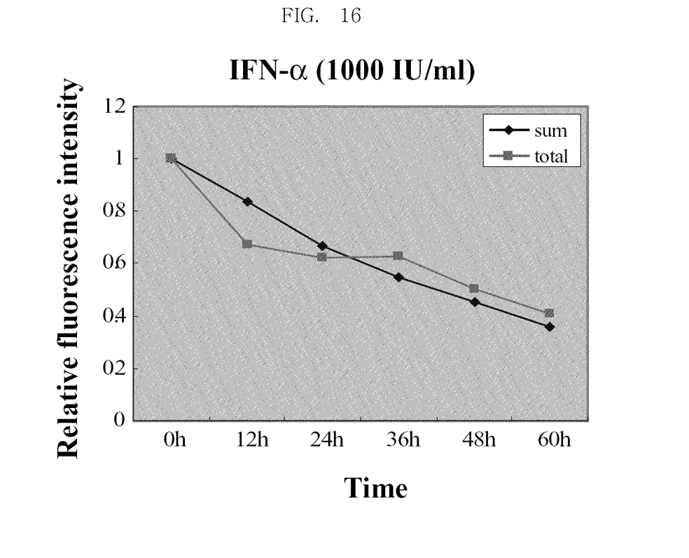

FIGS. 10 and 14 are graphs showing changing fluorescence levels in 8 transformants, in absolute values. FIGS. 11 and 15 are graphs showing changing fluorescence levels over time in 8 transformants, in relative values against the starting value, i.e., the value given to the transformants with no IFN-α treated. FIGS. 12 and 16 shows the graphs of relative value of the averaged fluorescence intensities of 8 transformants to the fluorescence intensities of the whole transformants changing over time, where the eight transformants were selected by each fluorescence intensity.

As demonstrated in FIGS. 9-16, in cells not treated with IFN-α, the total intensity of 5a-GFP fluorescence increased as cultivation time increased. In cells treated with IFN-α, seven among eight transformants showed decreasing fluorescence intensities over time. The increase or decrease in fluorescence intensities varied among the transformants.

From the results above, it is confirmed that the JFH 5a-GFP RNA permits real-time monitoring for the degree of living HCV replication, and provides a system for monitoring the anti-HCV effect in individual infected cells.

### Example 4

### Selecting Cells that are Permissive to Infection

We applied the consecutive two-fold dilution method into the Huh 7.5.1 cell line (Francis Chisari at Scripps Research Institute), which is known to be permissive to virus infection, to obtain a single cell to be cultured in a single wall. With a 96 well plate, the Huh 7.5.1 cells were diluted several times consecutively in half concentration.

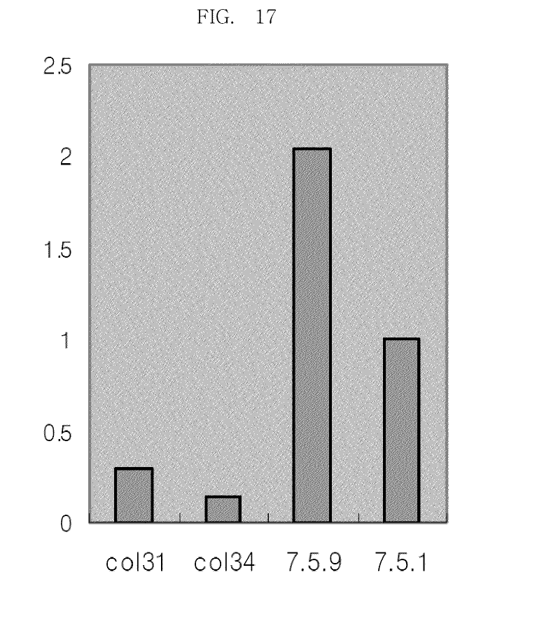

After obtaining 71 independent cell lines by cultivating cells in separate wells, each cell line was infected with the JFH 5a-Rluc virus, which permits a quantitative analysis of infection. The HCV-infected cells were cultivated in a Dulbecco's modified Eagle's medium with 10% fetal bovine serum at 37° C. under 6% of CO₂. Tissue culture 50% infectivity dose (TCID₅₀) was calculated by analyzing Renilla luciferase activities, as shown in FIG. 17.

As FIG. 17 indicates, a cell line showed more than two times higher HCV infectivity than Huh 7.5.1 cell line. The cell line was named “Huh 7.5.9.” Some cell lines showed decreased infectivity by 70 to 80 percent.

### Example 5

### Cloning of the JFH 5a-GFP Plasmid with Cell-Culture Adaptive Mutations

5-1: Amplification of the DNA Encoding Structural Protein with Cell-Culture Adaptive Mutations

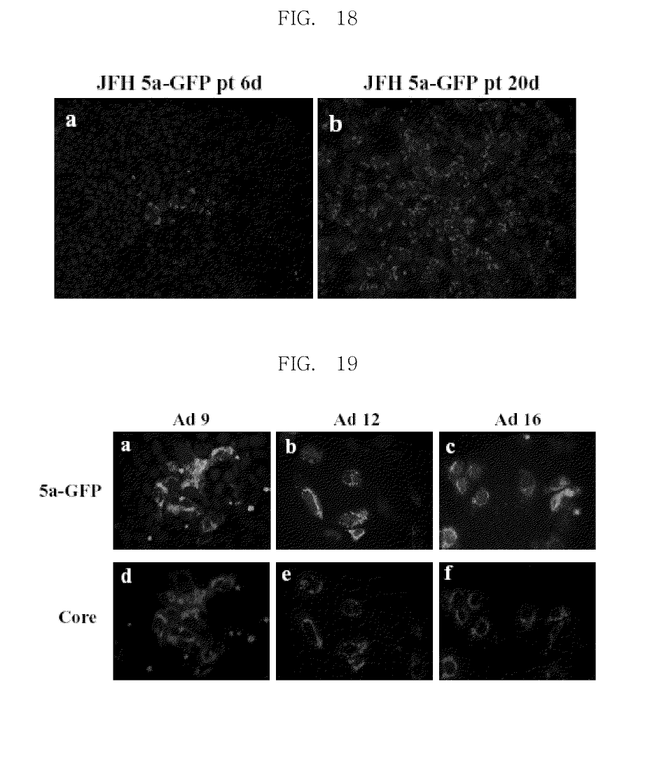

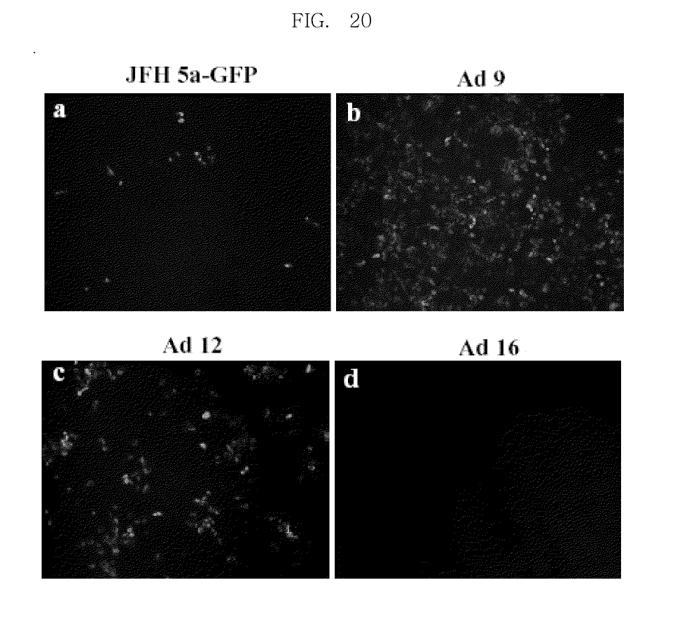

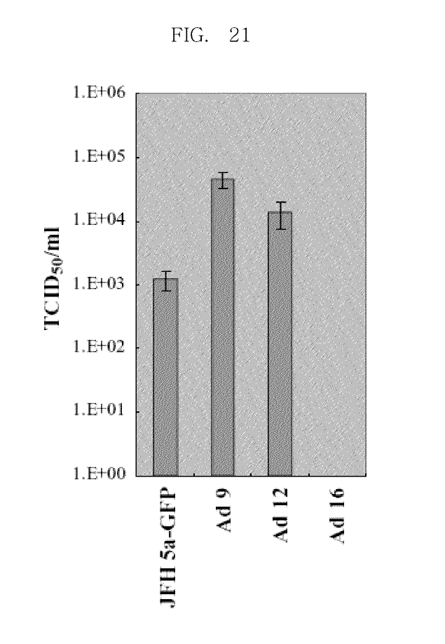

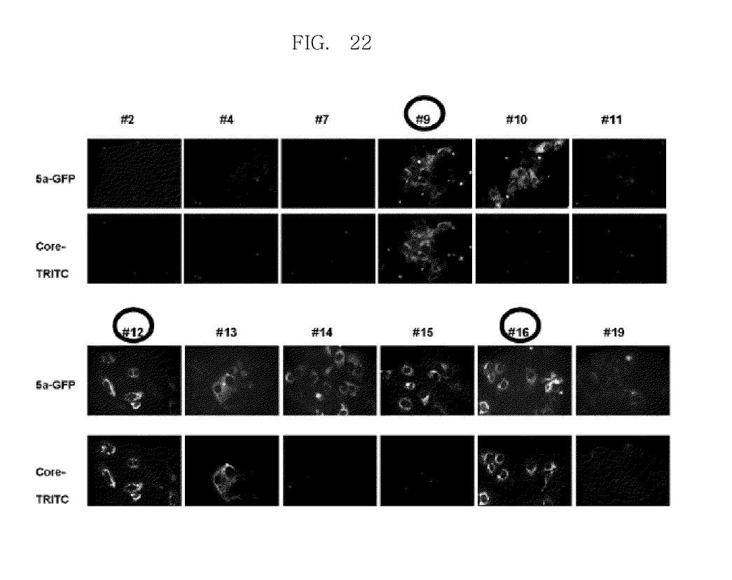

After being transfected with the JFH 5a-GFP RNA, the cells were cultured for 20 days in the medium and under the conditions as provided in Example 5-1. 20 days after transfection, the cell culture was collected and analyzed for infection, the results of which are shown in FIG. 18. By using the cultures collected 6 or 20 days post-infection, the Huh 7.5.9 cells were infected with the JFH 5a-GFP RNA. Expression of core protein was examined in the infected cells by using an immunocytochemical method using an antibody against the HCV core. The results are shown in FIGS. 19 to 21.

As shown in FIG. 18, almost all cells were effectively infected when the culture obtained from 20 day post-inoculation was used, whereas the culture obtained from 6 day post-inoculation resulted in infection of only a few cells. The results indicate that in the culture obtained from 20 day inoculation, adaptive mutations had accumulated, which enables highly efficient infection.

As shown in FIG. 19, among the clones which has substitution of a part of the JFH 5a-GFP, only three clones, i.e., Ad 9, Ad 12, and Ad 16, expressed core and NS5a-GFP proteins when transcripts of the clones were transfected into Huh 7.5.9 cells.

Total RNA was isolated from the cells infected with the culture. To identify adaptive mutations, a cDNA for structural proteins (from core to NS2 proteins) was manufactured via RT-PCR (reverse transcription PCR). cDNAs were generated by using the reverse primers in Table 3 and the isolated total RNA for 1 hr at 43° C., with the Expand reverse transcriptase (Roche Inc.), and then the cDNAs were amplified by PCR.

TABLE 3

| | | SEQ |
|---|---|---|
| | | ID |
| Name | Nucleotide sequence (5′→3′) | NO: |
| Reverse | 5′-CCGAGAGCACACAGCTG-3′ | 10 |
| transcription | | |
| primer (csp 426) | | |
| #3 forward | 5′-GCCTAGCCATGGCGTTAG-3′ | 11 |
| primer (csp 423) | | |
| #3 reverse | 5′-TCGGAAGAGCCCAACGAC-3′ | 12 |
| primer (csp 427) | | |

5-2: Preparation of the JFH 5a-GFP Plasmid Clone Containing Cell-Culture Adaptive Mutations; and RNA Synthesis

The DNA amplified in Example 6-1 was digested with restriction enzymes Avr II and Age I. This DNA was inserted into the JFH 5a-GFP treated with the same restriction enzymes to generate infectious HCV clones with adaptive mutation(s). 12 clones were found to have correct inserts. The 12 clones were digested with the restriction enzyme Xba I to prepare DNAs templates for RNA synthesis. The DNA templates for the RNA synthesis were extracted by phenol and then ethanol-precipitated. Subsequently, RNAs were synthesized by using the T7 RNA polymerase (Stratagene Inc.). After removing the template DNAs with DNase I (Ambion Inc.), the remaining RNAs were quantified using a UV spectrophotometer.

5-3: Generation of the Viruses from the JFH 5a-GFP with Adaptive Mutations; and Measurement of their Infectivity

The RNAs synthesized in Example 5-2 were transfected into cells by electroporation. Three days after transfection, the expressions of core and NS5a-GFP proteins in the cells were visualized by fluorescence microscopy, the results of which are shown in FIG. 18.

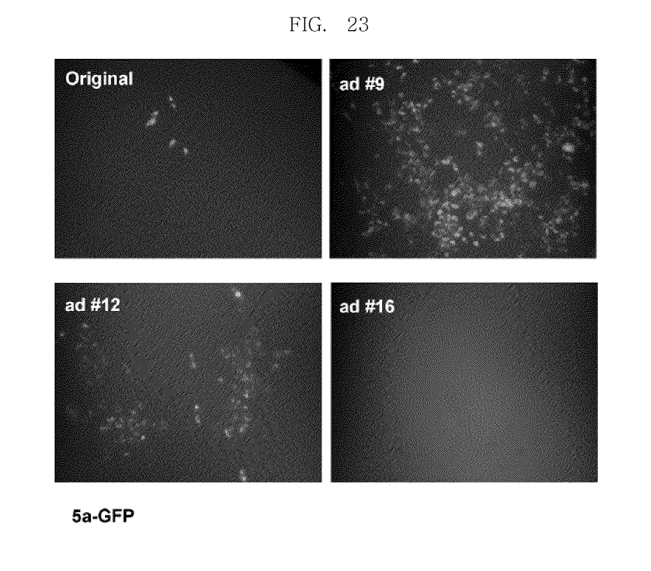

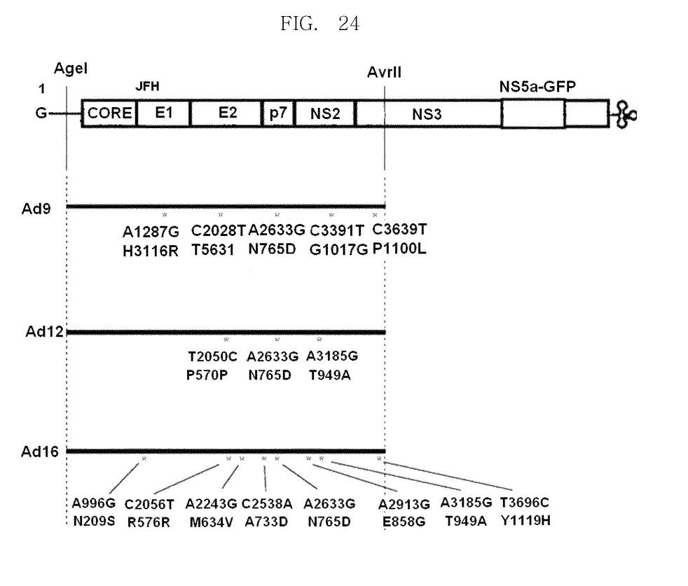

The RNAs synthesized in Example 5-2 were transfected into cells by electroporation. Seven days after transfection, the cell culture was harvested and was used to infect cells. The expressions of NS5a-GFP proteins in the cells were visualized by fluorescence microscopy to quantify infectivity, the results of which are shown in FIGS. 19 to 23.

As indicated in FIGS. 19 to 23, the virus from the # 9 clone—compared with the original virus—was found to have the highest infectivity, while the virus of the #12 clone showed a decent degree of infectivity. By contrast, no infection was observed from the #16 clone. The results indicate that cell-culture adaptive mutations in # 9 clone provide the highest infectivity of the virus among the viruses tested, while mutations in the #16 clone caused problems in virus infection.

### Example 6

### Isolation and Identification of Mutations Facilitating Virus Formation; Sequence Analysis of the Ad9, Ad12, and Ad16 Clones

6-1: Sequence Analysis of Ad9, Ad12, and Ad16 Clones

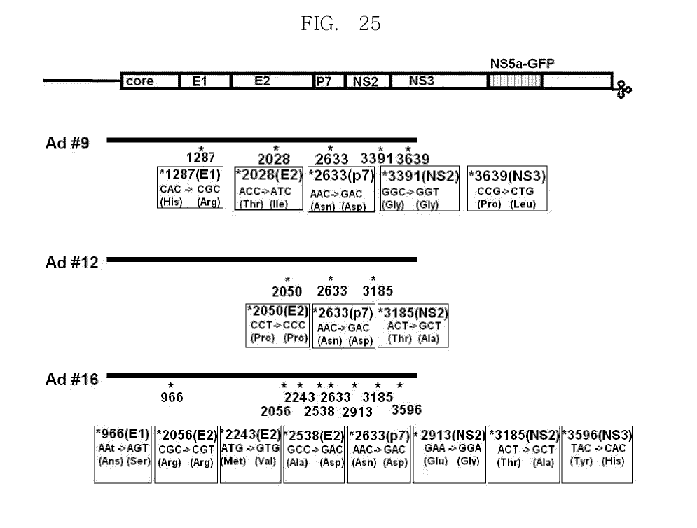

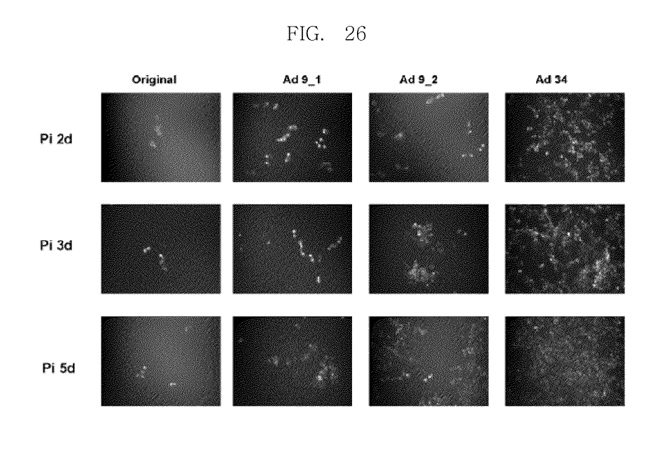

To identify base sequences altered in the Ad9, Ad12, and Ad16 clones of Example 5, their sequences were analyzed. The results are shown in FIGS. 24 and 25.

6-2: Identification of Critical Mutations Augmenting Virus Proliferation, Among the Various Changes in Bases of the Ad 9 clone.

The Ad9 clone (named JFH 5a-GFP ad#9, see FIG. 1) contained base changes at five points. To identify critical mutations augmenting virus proliferation, clones with each base change were prepared, and then their virus forming activities were analyzed. The results are shown in FIG. 26.

As indicated in FIG. 26, the change in the E2 protein (named JFH 5a-GFP ad#9_{—}1, see FIG. 1) and the change in the p7 protein (named JFH 5a-GFP ad#9_{—}2, see FIG. 1) were found to play important roles in the enhanced virus forming activity (Ad#9_{—}1 and Ad#9_{—}2, See FIG. 26). When the two mutations existed together (JFH 5a-GFP ad#34, See FIG. 26), virus-forming activity was greatly maximized.

1. A polynucleotide comprising a modified HCV genome being capable of effectively inducing infection which comprises at least one alteration in a nucleotide sequence selected from the group consisting of a nucleotide sequence encoding E2 protein and a nucleotide sequence encoding p7 protein in the RNA genome of a JFH1 strain shown in SEQ ID NO: 1.

2. The polynucleotide according to claim 1, wherein the alteration in the sequence encoding the E2 protein occurs at one or more nucleotide selected from the group consisting of nucleotides of 2027-2029 in the nucleotide sequence shown in SEQ ID NO:1, and the altered nucleotide sequences does not encode Threonine.

3. The polynucleotide according to claim 1, wherein the alteration(s) in the sequence(s) encoding the p7 protein occurs at one or more nucleotide selected from the group consisting of nucleotides of 2633-2635 in the nucleotide sequence shown in SEQ ID NO:1, and the altered nucleotide sequences does not encode Asparagine.

4. The polynucleotide according to claim 1, wherein the modified HCV genome further comprises a reporter gene inserted into NS5a protein-coding sequence in the RNA genome of a JFH1 strain shown in SEQ ID NO: 1.

5. The polynucleotide according to claim 4, wherein the reporter gene is selected from the group consisting of genes encoding Renilla luciferase, green fluorescence protein (GFP), firefly luciferase, red fluorescence protein (RFP), and secreted alkaline phosphatase (SeAP).

6. The polynucleotide according to claim 4, wherein the reporter genes are inserted right after nucleotide 7176, 7179, 7182, 7185, or 7188.

7. A modified HCV comprising the polynucleotide according to claim 1.

8. A cDNA of the RNA of modified HCV genome according to claim 1.

9. A vector comprising the polynucleotide according to claim 1.

10. The polynucleotide according to claim 9, wherein the vector is for a virus vector for hepatocytes.

11. A transformant comprising the polynucleotide according to claim 1.

12. A virus particle of polynucleotide of the modified HCV according to claim 1.

13. A virus particle of the modified HCV that is obtained from a culture in which the transformant according to claim 11 is cultured.

14. An HCV-infected cell that is infected by a virus particle according to claim 12.

15. A vaccine for HCV or an neutralizing antibody that is obtained by using virus particles according to claim 12 as an antigen, in whole or in part.

16. A screening method for an anti-HCV material, the method comprising a step of cultivating a cell that is incorporated the polynucleotide according to claim 1, in the presence of a test substance, and a step of assessing anti-HCV effect of the test substance.

17. The screening method for an anti-HCV material according to claim 16, wherein the anti-HCV effect of the test substance is assessed by detecting and/or quantitatively measuring virus particles or a nucleic acid of the modified HCV in the cell culture.

18. The screening method for an anti-HCV material according to claim 16, wherein the anti-HCV effect of the test substance is assessed by detecting and/or quantitatively measuring expression products of the reporter gene in the cell culture.

19. A method for preparing a virus vaccine by using a virus particle of claim 12 or a part thereof as an antigen.

20. A gene therapy method using the polynucleotide of claim 1, or using the HCV virus particles according to claim 12, in whole or in part.
